(19) United States
(12) Patent Application Publication
(10) Pub. No.: US 2009/0149862 A1
(43) Pub. Date: Jun. 11, 2009

(54) Guide pin for pedicle screw placement and method for use of such guide pin in spinal fusion surgeries

(75) Inventor: Yong Jung Kim, Fort Lee, NJ (US)

Correspondence Address: ABELMAN, FRAYNE & SCHWAB, 666 THIRD AVENUE, 10TH FLOOR, NEW YORK, NY, 10017, US

(73) Assignee: SYM PARTNERS, LLC

(21) Appl. No.: 12/001,254

(22) Filed: Dec. 10, 2007

(51) Int. Cl. A61B 17/58; A61B 17/56

(52) U.S. Cl. 606/104; 606/96; 606/329

(57) ABSTRACT

A radiopaque guide pin that is insertable into a pedicle of a vertebra to be used as a guide to determine the proper and safe entry point and angular trajectory for a pedicle screw placed in a pedicle, has a distal part and terminal tip for initial entry of the guide pin, an opposite proximal part that serves as a handle, and a central part that includes a first radiopaque marker spaced proximally from the tip and a stop element spaced proximally from the first marker, the stop element having greater diameter than the distal and central parts of the guide pin and adapted to be stopped by the lamina surface from further advancement into the pedicle. The invention further includes a method for pedicle screw placement using the new pedicle screw guide pin.

### I. FIELD OF THE INVENTION

The present invention relates to spinal fusion surgery and more particularly to an improved method for placement of pedicle screws in a pedicle and to a guide pin used in this new method to establish a safe and preferred entry point and angular orientation of the guide pins and of pedicle screws that avoid harm to the spinal cord or damage to the vertebra.

### II. BACKGROUND

There are many techniques and devices currently used by surgeons in spinal fusion surgeries. One method of fusing two or more vertebrae of the spinal column involves the use of screws which are inserted into the left and right pedicle portions of the vertebrae. Rods or other connectors are then attached to the pedicle screws of successive vertebrae in order to stabilize the positioning spacing and orientation of one vertebra relative to the next. Pedicle screw fixation is a very useful and frequently a necessary procedure in various types of spinal surgeries. Despite many advantages of using pedicle screws, their use in the spine involves danger with a potential for permanent neurologic deficit, especially when placing screws near the spinal cord at the concave apex of a scoliotic spine. In the literature there are numerous case reports that describe grave complications related to the erroneous placement of thoracic pedicle screws, such as cardiac tamponade and postoperative paraplegia.

Accurate placement of pedicle screws in the spine is essential to prevent serious complications, such as cardiac tamponade, aortic perforation, paraplegia and death. Correct starting and aiming points for accurate pedicle screw placement are difficult to obtain using currently available techniques other than stereotactic image guided systems. Several complications may arise if the pedicle screws are not located correctly. The two most important factors when inserting pedicle screws are (1) determining an accurate pedicle screw entry point and (2) establishing an accurate and safe trajectory or angular orientation of the pedicle screw through the pedicle isthmus.

There have been constant and continued efforts to develop methods to enhance the safety of pedicle screw fixation. Ideally, the entry point and the trajectory of the pedicle screw should be determined such that the pedicle screw placement will not violate the pedicle wall, and the center of the pedicle screw will generally follow the axis of the pedicle and pass through the center of the pedicle isthmus. Previous studies on the morphometry of pedicles, especially thoracic pedicles, indicate that there is a high inter-individual variability in the pedicle dimensions. The pedicles exhibited significant variability in their shape and orientation, not only between different vertebrae within the spine, but also in the same vertebra and even in the same pedicle among individuals. Therefore, the ideal entry point and the ideal trajectory for each screw should be determined individually and based on the accurate and available anatomical information of each vertebra.

Continued efforts to improve the safety of the pedicle screw fixation resulted in a few noticeable improved methods, that are represented by: (1) the method of using an intra-operative C-arm image intensifier that provides a useful tool for directly visualizing anatomic landmarks, i.e., the object pedicle with surrounding structures like the lamina, the transverse process, as well as the superior and inferior facet joints, (2) the method of placing K-wires into the pedicles to verify the entry point under fluoroscopy before preparing the hole for the screw (Myles R T et al. Spine, 1999), and as the most recent and accurate method, (3) stereotactic image guided systems based on a preoperative CT scan or fluoroscopy.

The methods (1) and (2) depend on two-dimensional radiographic images that typically consist of two views taken at approximately right angles to one another. However, these methods have been shown to result in significant differences in starting point location by individuals who perform surgeries. The stereotactic image guided systems based on preoperative CT scan or fluoroscopy (Method (3)) have been demonstrated to improve the accuracy of pedicle screw placement; however, this technique requires a preoperative CT scan with exposure to high levels of radiation, high cost, the need for expensive equipment and prolonged operation time. With all the efforts for improvement in pedicle screw fixation, the incidence of pedicle screw misplacement still remains high with the estimated range of 3% to 44.2% and pedicle screw-related neurologic complications occur in about 1% of total cases.

Repeated exposure to radiation is usually a necessity in determining the ideal starting point and the proper angle for pedicle screw placement. In actual clinical practices the actual trajectory of a fixation device is often inaccurate because there is no clear aiming point. The pedicle is not visualized during the operation. Moreover, since the bony tissue is hard and unyielding, the track that is made by the pin or the drill bit is dependent on the angle at which the pin or the drill bit initially approaches the surface of the bone and the starting point. The correct angle is difficult to determine under normal circumstances, and multiple attempts are often made to place the pin or the drill bit correctly with the aid of the feedback obtained from repeated fluoroscopic images. This process is repeated for each pedicle screw fixation, causing a high radiation exposure to both the patient and all personnel involved with this surgical procedure. This process is also highly time consuming since C-arm images are taken every time and the drapes are rearranged each time an image is taken. This exposure to radiation is considered to be a serious health hazard. Ionizing radiation has no safe threshold of exposure below which it ceases to have adverse effects, although an arbitrary level is assumed in clinical practice. A proper risk estimate of radiation exposure is not possible because absolute levels of safe exposure remain unknown and the effect of the accumulation of radiation exposure is not known. Thus, radiation exposure to the surgical team as well as to the patient during surgery continues to be an ongoing concern. A procedure that reduces the amount of radiation exposure would be highly desirable.

Many patents address issues related to pedicle screw fixation. Typically, the flesh adjacent to the backbone is opened to expose the spinal column, whereupon the location of the pedicle of the spinal bone is visibly determined. In some prior art disclosures a ball tip is used to examine the surface of the pedicle to determine presence of irregularities or cracks, which will reduce the integrity of the pedicle fixation. The publications in the prior art do not disclose a safe and relatively easy method to select an insertion point in the pedicle and to select insertion direction for a pedicle screw into the pedicle.

### III. OBJECTS AND SUMMARY OF THE NEW INVENTION

A first object of this invention is to provide a pedicle screw placement device that accurately positions a pedicle screw centrally within the bone mass of the pedicle, thereby preventing the pedicle screw from reaching too close to the nerve cells of the spinal column or being too close to the external surface of the bone, and thus to avoid cracks on the bone surface that could compromise the integrity of the pedicle screw fixation.

A related object is to provide a method for determining an ideal and safe insertion of entry point for the pedicle screw, and for determining an ideal and safe angular orientation or trajectory of the pedicle screw so that it will be positioned centrally within the bone mass of the pedicle.

A further object is to provide a method of pedicle screw placement that is a relatively simple routine technique that less experienced professionals can employ safely.

An additional object is to provide a method of pedicle screw placement that requires only conventional x-ray radiographic equipment with the fewest possible exposures of x-ray radiation to the patient and to medical staff.

An additional object of this invention is to provide a method of pedicle screw placement which requires only lateral and vertical x-ray radiographs to determine the desired safe entry point and angular orientation of the pedicle screw.

Another object is to provide a method for a pedicle screw to be advanced along a pathway generally close to the center of the isthmus of the pedicle.

It is a further object for the new pedicle screw placement device to be a single thin, elongated radiopaque guide pin of predetermined length with at least one radiopaque marker situated at a predetermined distance from its distal end so that from X-ray radiographs a surgeon can determine if the trajectory is safe and can determine the preferred length and diameter for the pedicle screw.

It is still a further object for the new guide pin of this invention to have a stop element situated proximally from said marker and extending radially outward a distance greater than the guide pin diameter, the stop element provided to engage the lamina at the pedicle entry point and restrain further pedicle guide pin advancement.

It is thus an object of the present invention to provide a device and method for pedicle screw placement that improves the accuracy of positioning a pedicle screw into the pedicle of a vertebra as part of a spinal fusion procedure.

A preferred embodiment of the new device of this invention is a generally stiff, thin elongated radiopaque guide pin, preferably of a metal such as a titanium alloy or 304 stainless steel, that is provided at predetermined lengthwise locations with one or more axially spaced radiopaque markers and a stop element. For convenience and clarity of terminology herein, the new guide pin, while usually being a single continuous wire, will be described as having a proximal portion that is held and manipulated by the surgeon, an opposite distal portion including a terminal end or tip that is directed and advanced into the pedicle, and a junction area or central portion between said proximal and distal portions.

A. Summary of Pedicle Screw Guide Pin Embodiments

Preferred embodiments of the new guide pin have the following configuration variations:
(a) a basic embodiment is a radiopaque pin or rod of predetermined length L, and having a proximal portion serving as a handle, an opposite distal portion terminating in a tip end, a junction or central area between said proximal and distal portions, and at least one radiopaque marker on said distal portion located a predetermined distance proximally from said distal portion tip,
(b) an embodiment like (a) but having a second radiopaque marker spaced a predetermined distance proximally from the first marker.
(c) an embodiment like (a) but having a stop element that has greater diameter than that of said distal portion and is located a predetermined distance proximally from said first marker and between said first marker and said proximal end,
(d) an embodiment like (c) but having a second marker located between said first marker and said stop element,
(e) an embodiment like (a) but having a stop element of (c) instead of a marker located a predetermined distance from said distal end, and
(f) an embodiment where a distal portion having a first diameter is connected to a central part having a larger diameter which is connected to a proximal portion that serves as a handle, with a stop element at the junction of said proximal and central parts.

In said various embodiments of guide pins the tip of said distal portion of the guide pin, whether it is rounded or sharp, is adapted to be easily inserted into an entry point of the pedicle bone. The cross-sectional shape of this distal portion may be circular, oval, elliptical, rectangular or any suitable cross-section of diameter of about 1.5 to 3 mm, and the length may be up to about 20 cm, and preferably about 2 cm. The distal portion may be tapered in the proximal direction to have a larger diameter for easier penetration of the guide pin and for improved x-ray radiograph image visibility. Each radiopaque marker is typically a spherical section to allow ease of insertion and to improve guide pin x-ray image visibility as it extends through the pedicle and facilitates identification of the pedicle isthmus. These guide pins are relatively stiff and may be made of any radiopaque material, examples including stainless steel, preferably 316 L or 304, titanium, titanium composites or titanium alloy, or mixtures of the above.

In embodiments (c) through (f) described in Paragraph 0020 above, the stop element is an X-shaped marker formed of a pair of metallic wires having length of up to about 8 mm, and welded or integrally formed with the guide pin, and designed to limit the insertion depth of said distal portion into the pedicle bone. During insertion of the guide pin the X-shaped marker engages and stops on the lamina of the vertebra, and in the x-ray radiographic image this X-shaped stop element indicates the location of the insertion point of the guide pin into the pedicle bone.

The proximal section extends proximally from the X-shaped marker, serves as a handle to insert the distal section into the pedicle bone, and preferably is co-axial with said distal section to readily indicate the angle of penetration of the guide pin within the pedicle. The guide pin is inserted into the pedicle until the X-marker contacts and is stopped by the lamina. In this state the X-shaped marker is visible to the surgeon on the pedicle bone surface of the patient, while the distal section is submerged in the pedicle bone structure and therefore invisible to the surgeon without X-ray radiographs. As described earlier, lateral and vertical or other radiographs are now taken to indicate the actual orientation of the guide pin in the pedicle.

When the guide pin is initially placed said central section or transition point and X marker will be at the lamina/entry point. The length of the selected pedicle screw will be sufficient to extend fully through the pedicle and into the body of the vertebra; the diameter of the pedicle screw will be appropriate for the isthmus of the pedicle.

After the pedicle screws are installed in each of the two, three or more adjacent vertebrae, left and right fixation rods are attached to the exposed heads of the emplaced pedicle screws in a known conventional manner, and conventional closure of the wound follows.

B. Summary of the Method of Pedicle Screw Placement with the New Guide Pin

In accordance with the new method, after the guide pins are initially placed the patient is taken for x-ray radiography where two orthogonal radiographs are taken, typically one radiograph providing a vertical image and a second radiograph providing a lateral horizontal image, producing posteroanterior and lateral x-ray images. The radiographs image both the interior structure of the pedicle bone and the inserted guide pin with its first, second sections as well as the X-shaped marker stop element. The X-shaped marker represents exactly the guide pin insertion point at the pedicle bone surface of the patient, while the imaged guide pin first and second sections and any radiopaque markers thereon show how the guide pin is oriented within the interior pedicle bone structure of the patient. With this procedure it is easy to observe if the inserted tip of the guide pin is too close to the spinal canal of the pedicle or too close to the external surface of the pedicle bone surface. If the pedicle screw inserted at the guide pin were to touch the spinal cord, nerve damage could result resulting in paralysis or other complications. Similarly, if the pedicle screw intersects the external surface of the pedicle bone, cracks could form compromising the integrity of pedicle screw fixation process. Ideally, the guide pin should be located on a centerline between these two angular extremes. The surgeon observes this centerline orientation and any angular misalignment in the vertical plane and horizontal plane by studying the two X-ray images. If misalignment exists the surgeon withdraws the guide pin and reinserts it in a new selected angular orientation, preferably still using the same entry point, and then produces two additional orthogonal radiographs to confirm the angular re-orientation of the guide pin, which is now located in the desired centerline of the pedicle bone structure. The length of the pedicle screw needed is easily estimated using radiography to compare the known distances between markers and the observed bone structure.

After a determination that the guide pin has a desired entry point, orientation and centrally located position within the pedicle, the surgeon may select from a number of different final steps as follows:
a) Removal of the guide pin and placement of a pedicle screw;
b) Removal of the guide pin, insertion of a gearshift awl or spear to enlarge the passageway formed by the guide pin, removal of the awl followed by the placement of a pedicle screw;
c) Removal of the guide pin, insertion of a gearshift awl to enlarge the hole, withdrawal of the gearshift awl, followed by the insertion of a tap/drill to establish threads in the walls of the hole, removal of the tap/drill, and the final placement of a pedicle screw; or
d) While a guide pin remains emplaced, (i) inserting coaxially over said guide pin a cannulated tap/drill with external threads to cut threads in the walls of the passageway formed by said guide pin, (ii) withdrawing the tap/drill and guide pin, and then (iii) inserting the pedicle screw. For this last step the guide pin must be of the type without an X-shape stop element or with the legs of the X-shape stop element removed.
e) After withdrawal of a guide pin the surgeon may palpate the passageway walls with a flexible ball-tipped pedicle sounding or palpating device to confirm that the walls of the passageway have not gotten too close or broken through the pedicle periphery. In the initial placement of the guide pin the lamina may be slightly penetrated by a round burr or other drill to establish a starting location for entry of the guide pin.

### IV. BRIEF DESCRIPTION OF THE DRAWINGS

The invention will be more fully understood and further advantages will become apparent when reference is had to the following detailed description and the accompanying drawings, in which:

FIG. 1 is a fragmentary side elevation or lateral view of lumbar vertebrae of a human spine,

FIG. 2 is a fragmentary rear or posterior view of the third and fourth lumbar vertebrae shown in FIG. 1,

FIG. 3 is a top plan view of a second lumber vertebra of the vertebrae shown in FIG. 1,

FIG. 4 is a top plan or superior view of a first embodiment of the new pedicle screw guide pin,

FIG. 4A is a side elevation or lateral view thereof,

FIG. 5 is a top plan or superior view similar to FIG. 1 of a second embodiment of the new pedicle screw guide pin,

FIG. 6 is a top plan or superior view of a third embodiment of the new pedicle screw guide pin; the side elevation view is substantially the same as the top plan view,

FIG. 7 is a top plan or superior view of a fourth embodiment of the new pedicle screw guide pin; the side elevation view is substantially the same as the top plan view,

FIG. 8 is a top plan or superior view similar to FIGS. 3 and 4 showing placement of the pedicle screw guide pins in a vertebra,

FIG. 8A is a fragmentary top plan view of a fifth embodiment of a new pedicle screw guide pin showing its X-shaped stop element with its legs extending radially outward and perpendicular to the axis of the guide pin,

FIG. 8B is an end elevation view of the guide pin of FIG. 8A,

FIG. 8C is a fragmentary lateral view similar to FIG. 4A, showing the first embodiment of the X-shaped stop element having its legs and the guide pin in the same plane,

FIG. 8D is an end elevation of the guide pin of FIG. 8C,

FIG. 8E is a fragmentary top plan view of a sixth embodiment of a new guide pin, showing its X-shaped stop element with its legs in a plane parallel to the plane of the guide pin,

FIG. 8F is an end elevation view of the guide pin of FIG. 8E,

FIG. 8G is a fragmentary top plan view of a seventh embodiment of the new guide pin, showing an enlarged ball-shaped stop element,

FIG. 8H is an end elevation view of the guide pin of FIG. 8G,

FIG. 9 is a simulated vertical (superior/anterior) radiograph showing position and angular orientation of a guide pin in a pedicle,

FIG. 10 a simulated vertical (superior/anterior) radiograph showing position and angular orientation of a guide pin in a pedicle,

FIG. 11 is a simulated radiograph of a top plan or superior view similar to FIGS. 3, 4 and 7, showing pedicle screw placement, and

FIG. 12 is a simulated radiograph of a fragmentary side elevation or lateral view of the pedicle screw placement of FIG. 9.

### V. DETAILED DESCRIPTION OF THE PREFERRED EMBODIMENTS

The features of the invention will become apparent from the following description of the exemplary embodiments taken in conjunction with the accompanying drawings. For convenience and clarity in describing these embodiments similar elements or components appearing in different figures will have the same reference numbers.

For purposes of this application, the word radiograph will be understood to mean not only conventional x-ray imaging seen as electronic projections and/or film, but also all forms of internal imaging of otherwise opaque items, including but not limited to electromagnetic radiation, x-ray, fluoroscopy, CT scan, MRI and ultrasound.

FIGS. 1-3 are prior art illustrations showing fragmentary views of a human spine, which establish a background disclosure of the location of pedicles on a typical vertebra for understanding the present invention.

FIG. 1 is a fragmentary side elevation view of a human spine 1, including five typical lumbar vertebrae 2.

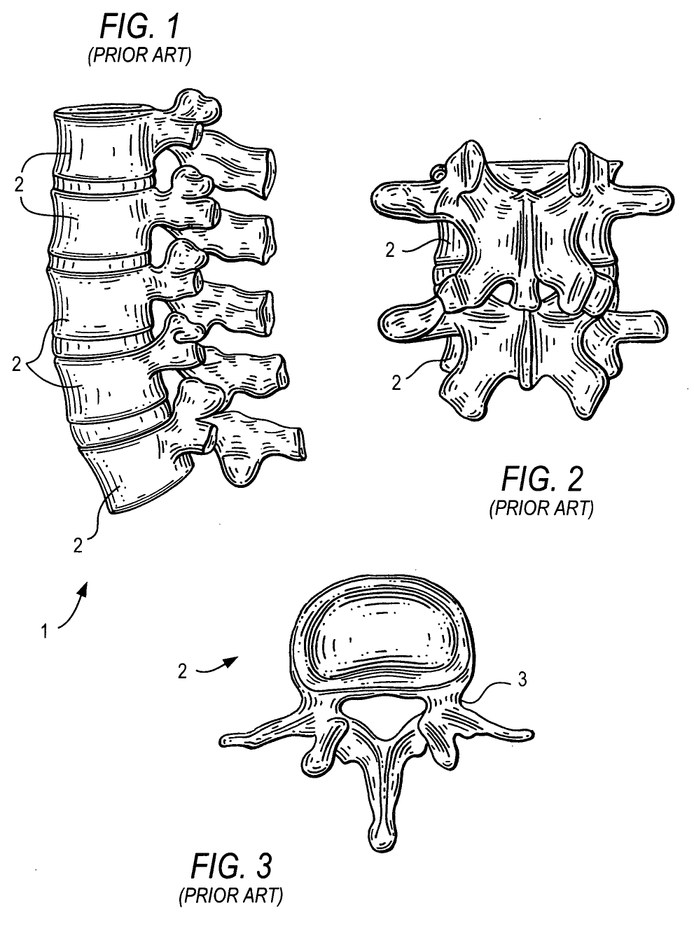

FIG. 2 is a fragmentary posterior or rear elevation view of the spine of FIG. 1, showing only two of the lumbar vertebrae.

FIG. 3 is a top plan view of the second lumbar vertebra of the spine of FIGS. 1 and 2, showing, in particular, the portion 3 identified as the “pedicle” which is the structure addressed by the present invention.

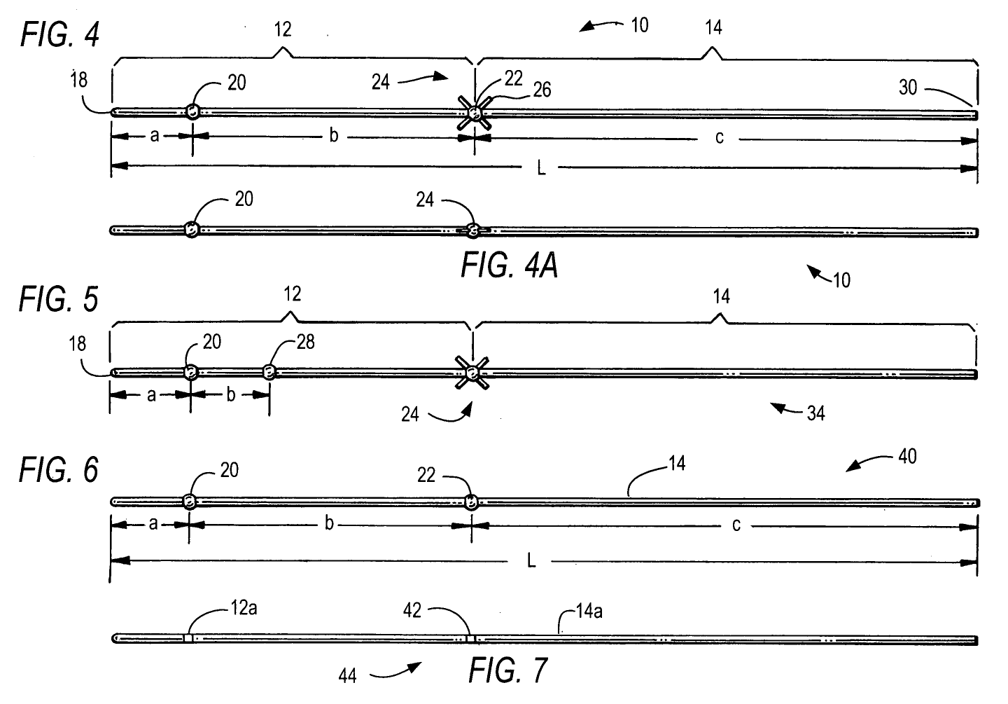

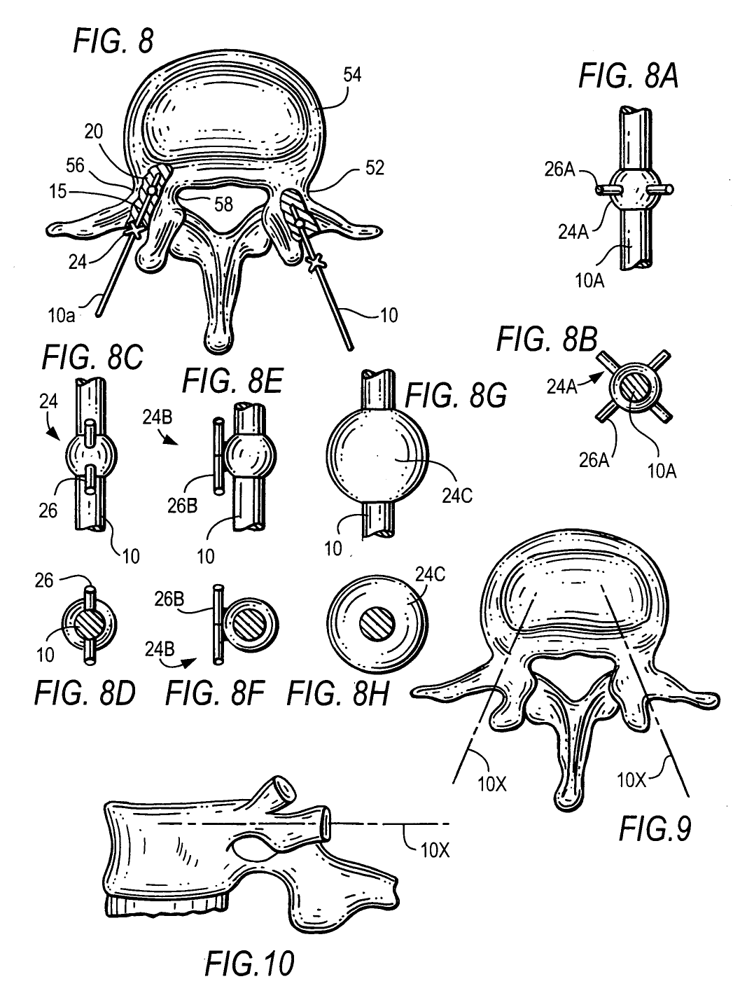

FIGS. 4-7 illustrate various embodiments of the new pedicle screw guide pin, and FIGS. 8-12 illustrate a preferred new method of pedicle screw placement using the new guide pin. For convenience and ease of understanding, similar components of different embodiments may be given the same reference numbers.

FIGS. 4 and 4A show guide pin 10 in top plan (superior) and side elevation (lateral) views respectively. Guide pin 10 is a relatively stiff radiopaque rod or wire having diameter of about 1-5 mm. and overall length L of about 10-40 mm.

This guide pin has distal portion 12, proximal portion 14 and central portion 16. These portions may comprise a single rod or wire, or may be separate components fixedly connected at junction areas. Proximal portion 14 serves as the handle held by the surgeon; distal portion 12, having tip end 18, is inserted via an entry point into and through the pedicle and ideally through the center of the pedicle's isthmus and into the body of the vertebra. The distal portion may taper in the proximal direction to a larger cross section for ease of insertion of the guide pin.

Guide pin 10 has a first marker 20 formed as a radiopaque sphere of diameter slightly greater than the proximal section rod diameter for easier identification in a radiographic film. Marker 20 is situated proximally from tip 18 by a distance a in the range of about 1-20 mm.

Guide pin 10 has at its central portion 16 a marker 22 similar to marker 20 and/or a stop element 24, where the marker is readily identifiable in a radiograph and the stop element, having a greater cross-section than proximal section 12 is adapted to engage the lamina and bar further axial advance of the guide pin.

Stop element 24 is formed by legs 26 forming an X-shape that extends radially outward. The stop element may have many other cross-sectional shapes, so long as the stop element extends radially outward farther than the cross-section of the proximal portion.

As seen in FIG. 5, distal portion 12 may have two generally similar markers 20 and 28. The distances from tip end 18 to marker 20, and between markers 20 and 28, and from marker 28 to stop element 24, are known. Thus, a radiograph showing these markers within a pedicle can indicate the necessary length required for the pedicle screw, and can indicate other dimensions within the pedicle and vertebra.

In guide pin embodiments shown in FIGS. 4-5 and 6 the cross-sectional shapes of sections 12 and 14 may be the same or different and may include round, square, rectangular, oval or other shapes. Where sections 12 and 14 are contiguous portions of a single wire or rod, there will be no individual junction areas except for the presence of markers 20 and 24 which may be enlargements formed in the rod or welded or otherwise fixed to the rod. In a later-described embodiment, legs 26 of stop element 24 may be broken off to allow a hollow-tube tap drill to be slid lengthwise over guide pin 10.

Handle or proximal portion 14 of guide pin 10 extends from central area stop element 24 or marker, proximally to proximal end 30 of guide pin 10. This handle portion 14 is illustrated as straight and coaxial with central portion 14 and distal portion 12, for engagement by the surgeon or by a tool held by the surgeon.

The material of guide pin 10 is preferably titanium or stainless steel, as described earlier. Markers 20 and 24 and stopper element 26 may be of the same material or of any radiopaque material that can be securely adhered to the basic guide pin rod material.

FIG. 5 illustrates a second embodiment 34 of a guide pin, which differs from guide pin 10 of FIG. 4 only by the additional marker 28. The distance b between markers 20 and 28 will vary depending on the dimensions of the vertebra and the surgeon's needs.

FIG. 6 illustrates a third embodiment 40 of a guide pin which is generally similar to guide pin 10 of FIG. 4, but has markers 20 and 24 without any further stop element enlargement.

FIG. 7 illustrates a fourth embodiment of a guide pin 44 with markers not shown, but comprising sections 12a and 14a which are fixed together at junction area 42, and may have different and/or tapering diameters along their lengths, or may each have constant diameters with a taper in a junction area.

As seen in FIGS. 4-1, there are numerous preferred guide pin embodiments and possible variations of same, as regards the number and axial positions of radiopaque markers and the use of a stop element between the distal and proximal sections of the guide pin. The distal and proximal sections' diameter is in the range of about 1-5 mm, preferably 1.5-3 mm. The distal section length is in the range of about 1-20 cm. The proximal section length is in the range of about 2-20 cm, preferably 5-10 cm. The stop element X-shape wires or bars have length in the range of about 2-8 mm and diameter in the range of about 1-4 mm. Notwithstanding the dimensions listed above for preferred embodiments, the guide pin invention disclosed herein is not limited to specific dimensions or materials.

FIG. 8 illustrates on the right side, a guide pin 10 (as seen in FIG. 1) partially inserted into a pedicle 52 of a vertebra 54. On the left side is another guide pin 10A fully advanced into pedicle 56, with stop element 28 barred from further advancement at the entry point in the lamina, and with a central section 15 of the guide pin ideally orientated centrally of the isthmus 58 of pedicle 56, the location of section 14 being identified and verified by marker 20.

The axial positioning of central section 15 and distal section 12, and the angular orientation thereof relative to the pedicle is determined by a set of vertical and horizontal X-ray radiographs. With reference to the guide pin of FIG. 5, for example, since distances a from the distal tip 18 to first marker 20, and b from marker 20 to marker 28, are known when the guide pin is selected for insertion, a surgeon can easily calculate from the X-ray radiographs the proper length of pedicle screw to use. Also, since the diameters of the guide pin sections 12 and 14 and of markers are known, the surgeon can see from the radiographs the radial distance to the external walls of the pedicle and calculate the proper diameter of pedicle screw to employ.

FIGS. 8A-8H illustrate four different embodiments of stop elements corresponding generally to stop element 24 seen in FIGS. 4 and 4A. FIGS. 8A and 8B show X-shaped stop element 24A with its legs 26A extending radially outward and perpendicularly to the central longitudinal axis guide pin 10A.

FIGS. 8C and 8D show more clearly the X-shape stop element 24 on guide pin 10.

FIGS. 8E and 8F show the sixth stop element embodiment 24B where the X-shaped legs 26B and guide pin 10 lie in generally parallel planes.

FIGS. 8G and 8H show a seventh stop element embodiment 24C in the form of an enlarged sphere about pin 10. Each of these stop elements can serve to restrain further axial advancement of the guide pin after such stop element engages the lamina. Also, these radiopaque stop elements are easily visible in the X-ray radiographs. As noted elsewhere, in selected embodiments of stop elements the legs of the X portion may be broken off so that while a guide pin remains inserted in a pedicle, a tubular tap drill can be slide axially over the guide pin and into the pedicle. In these situations the legs of the stop element served their purpose of stopping further axial advancement, and said legs are then removed to allow the next stage of tap drill advancement.

FIGS. 9 and 10 are simulations of radiographs which show the position and angular orientation of a guide pin represented by line 10× in a pedicle. As discussed herein, these radiographs either confirm correct and safe placement of the guide pin or else indicate the degree of improper placement and provide a basis for determining a correction. These radiographs also help the surgeon determine the distance from the lamina (where the stop element is) to the isthmus and into the body of the vertebra because of knowing the lengths of different portions of the guide pin.

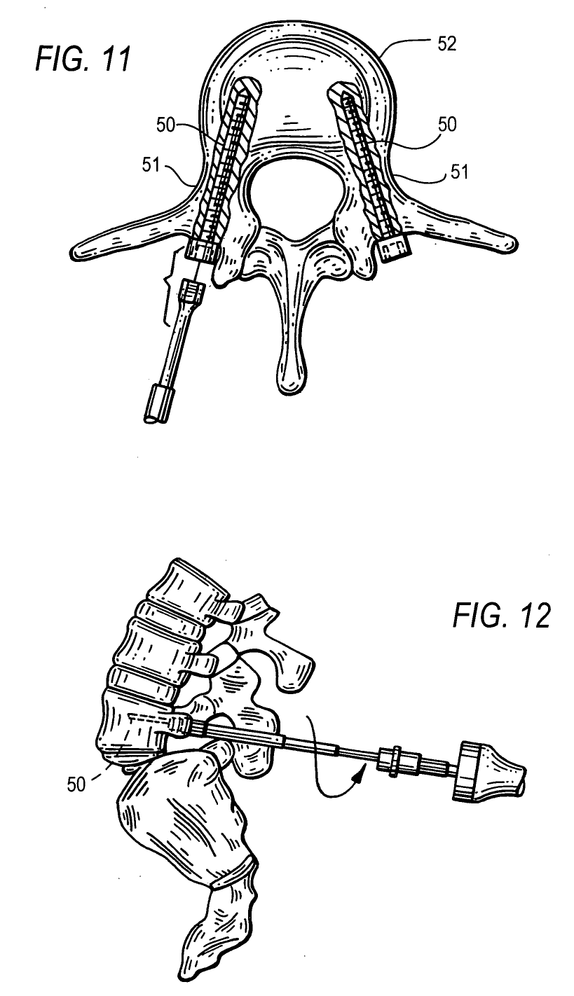

FIGS. 11 and 12 are further simulated radiographs showing placement by a screw driver tool of pedicle screws 50 in pedicles 51 of a vertebra, at the conclusion of this phase of the new procedure.

The new method, in summary, comprises:

1. selecting a starting point for insertion of a guide pin of the pedicle screw placement device into the pedicle after opening out the back of a patient;

2. inserting the distal portion (which may include the first and the second parts) of the guide pin, until the X-shaped stop element of the guide pin rests on the lamina bony surface of the vertebra;

3. taking x-ray radiographs of the patient in two orthogonal orientations, preferably vertical and horizontal directions to image the guide pin and the interior bone structure of the pedicle bone;

4. determining the angular orientation needed to place the guide pin in the centerline of the pedicle bone structure by observing in the radiograph the guide pin and its markers relative to the pedicle isthmus;

5. if necessary, determining corrective angular orientations of the guide pin relative to the pedicle and then withdrawing and reinserting the guide pin in the desired orientation to position said guide pin in the centerline of pedicle bone structure;

6. verifying safe and proper orientation of the guide pin by two additional orthogonal radiographs;

7. determining the proper length of the pedicle screw needed from the distance between the X-shaped marker and the insertion end of the guide pin corroborated with distance measurements from x-ray radiographs;

8. enlarging the passageway created by the guide pin by inserting a drill over the guide pin or inserting a drill through the guide pin passageway after the removal of the guide pin;

9. withdrawing the guide pin and drill or guide pin alone; and

10. inserting a pedicle screw of the required calculated length into the pedicle bone after tapping thread therein.

While the invention has been described in conjunction with several embodiments, it is to be understood that many alternatives, modifications, and variations will be apparent to those skilled in the art in light of the foregoing description. Accordingly, this invention is intended to embrace all such alternatives, modifications, and variations which fall within the spirit and scope of the appended claims.

1. A guide pin insertable into a pedicle of a vertebra to be used as a guide to determine an initial entry point and entry orientation angle of a pedicle screw, the guide pin comprising:
a radiopaque pin having a distal tip adapted to be inserted into a lamina and pedicle of a vertebrae; and
a radiopaque stopper disposed on the pin at a selected distance from the distal tip and adapted to abut the exterior surface of a lamina of the vertebrae.

2. A guide pin according to claim 1, further comprising a handle extending from the radiopaque stopper in a proximal direction.

3. A guide pin according to claim 1, further comprising a radiopaque marker disposed between the distal tip and the radiopaque stopper.

4. A guide pin according to claim 1, wherein the distance between the distal tip and the stopper is in a range of 1 to 20 mm.

5. A guide pin according to claim 1, wherein the diameter of the guide pin is in a range of 1 mm and 3 mm.

6. A guide pin according to claim 1, wherein the stopper is in the shape of an X.

7. A guide pin according to claim 1, wherein the stopper is at least partially breakable from the guide pin.

8. A guide pin insertable through the lamina and into pedicle of a vertebra, to be used as a guide for pedicle screw placement in said pedicle, comprising:
a. an elongated radiopaque rod of predetermined length L, said rod having a distal portion terminating in a tip end and an opposite proximal portion, and
b. a radiopaque stop element on said rod located a predetermined distance dl proximally from said tip end, said stop element adapted to engage said lamina when said rod is inserted therein and to restrain said rod from further axial advancement into said pedicle.

9. A guide pin according to claim 8, further comprising at least one radiopaque first marker on said distal portion of said rod, located axially between said tip end and said stop element, said first marker having dimensions such that it can be visibly distinguished from said distal portion in a radiograph of said rod and said first marker.

10. A guide pin according to claim 9, wherein said distal portion extends axially, and said first marker extends transversely of said distal portion.

11. A guide pin according to claim 10, wherein said distal portion where said first marker is axially located has a cross-sectional configuration that extends transversely of said rod, and said first marker has a cross-sectional configuration, at least one portion of which extends transversely further than said cross-sectional configuration of said distal portion, for enhancing visualization of said first marker relative to said rod in a radiograph.

12. A guide pin according to claim 8, wherein said rod extends axially and has a cross-sectional configuration where said stop element is axially located, and said stop element has at least one part that extends transversely outward a distance greater than said cross-section of said rod.

13. A guide pin according to claim 9, wherein said rod has a generally round cross-sectional configuration of diameter D, and said first marker has diameter greater than D.

14. A guide pin according to claim 8, wherein said distal portion has a predetermined cross-sectional configuration, said proximal portion has a similar but larger cross-sectional configuration, and said rod includes a tapered junction area between and connecting said distal and proximal portions.

15. A method of determining an initial entry point and entry orientation angle of a pedicle screw to be inserted into a vertebra using a guide pin including a radiopaque pin having a distal tip and a radiopaque stopper disposed on the pin at a selected distance from the distal tip and adapted to abut the exterior surface of a lamina of the vertebra, the method comprising:
inserting the distal tip of the guide pin through the lamina, pedicle and isthmus of the vertebra until the radiopaque stopper abuts the lamina;
imaging the area around the inserted guide pin to visualize the angular orientation and entry point of the inserted guide pin; and
based on the imaged area, determining an entry point and angular orientation of a pedicle screw to be inserted into the lamina.

16. A method according to claim 15, wherein the step of imaging includes taking orthogonal radiographs of the area around the inserted guide pin.

17. A method for pedicle screw placement in a patient's pedicle, using a guide pin formed as an elongated radiopaque rod of predetermined length L, said rod having a distal portion terminating in a tip end and an opposite proximal portion and a radiopaque stop element on said rod located a predetermined distance dl proximally from said tip end, said stop element having a cross-sectional configuration different from that of said distal portion of said rod where said stop element is axially located, such that at least part of said stop element extends radially outward further than the corresponding part of said cross-sectional configuration of said rod, said method comprising the steps:
a. directing said tip end of said guide pin into an entry point in the lamina of the vertebra and advancing said distal portion of said guide pin into the pedicle and into the isthmus thereof until said stop element engages the lamina and restrains said guide pin from further axial advancement,
b. taking orthogonal radiographs of said guide pin in said vertebra to visualize the angular orientation and distance of axial advancement of said guide pin within said pedicle, and
c. from said radiographs determining a desired and safe entry point and angular orientation for a pedicle screw to be inserted into said pedicle.

18. A method according to claim 17, wherein determining said angular orientation of said guide pin comprises observing in said radiographs the trajectory of said distal portion of said guide pin relative to the boundaries of the isthmus.

19. A method according to claim 17, wherein determining said axial advancement of said guide pin comprises comparing the known distance from the stop element to the tip end of the distal portion of the guide pin with the visualized axial location of the tip end in the isthmus.

20. A method according to claim 17, comprising the further steps of
a. withdrawing the guide pin from the passageway formed by said guide pin,
b. advancing a pedicle screw into said passageway, and
c. taking a further set of orthogonal radiographs to confirm desired and safe placement of the pedicle screw.

21. A method according to claim 20, comprising, subsequent to said step a of withdrawing said guide pin, the further step of enlarging said passageway by inserting a tapered awl of greater cross-section than that of said guide pin, and then removing said tapered awl.

22. A method according to claim 21, comprising the further step, after removal of said awl, of inserting a tap drill to form screw threads in the walls of said enlarged passageway.

23. A method according to claim 17, comprising the further step of enlarging said passageway by inserting coaxially over said guide pin still within pedicle, a hollow tap drill to form screw threads in the walls of said passageway, and then withdrawing said guide pin and tap drill.

24. A method according to claim 20, after placement of two pedicle screws in the left and right sides respectively of each of at least two adjacent vertebrae, whereby said left and right side pedicle screws are generally aligned respectively, the further step of securing said left side pedicle screws to a left side connecting rod, and similarly securing said right side pedicle screws to a right side connecting rod, which connecting rods fix the axial distances between adjacent connected vertebrae and stabilize the relative orientation thereof.
